(19) United States
(12) Patent Application Publication
(10) Pub. No.: US 2014/0051184 A1
(43) Pub. Date: Feb. 20, 2014

(54) MOBILITY SHIFT ASSAYS FOR DETECTING ANTI-TNF ALPHA DRUGS AND AUTOANTIBODIES THERETO

(71) Applicant: NESTEC S.A., Vevey (CH)

(72) Inventors: Sharat Singh, Rancho Santa Fe, CA (US); Scott Hauenstein, San Diego, CA (US); Linda Ohrmund, San Diego, CA (US)

(21) Appl. No.: 13/797,815

(22) Filed: Mar. 12, 2013

Related U.S. Application Data
(60) Provisional application No. 61/683,681, filed on Aug. 15, 2012.

(51) Int. Cl. G01N 33/537

(52) U.S. Cl. CPC G01N 33/537
USPC 436/501

(57) ABSTRACT

The present invention provides assays for detecting and measuring the presence or level anti-TNFα drugs and/or the autoantibodies to anti-TNFα drugs in a sample. The present invention is useful for optimizing therapy and monitoring patients receiving anti-TNFα drug therapeutics to detect the presence or level of autoantibodies against the drug. The present invention also provides methods for selecting therapy, optimizing therapy, and/or reducing toxicity in subjects receiving anti-TNFα drugs for the treatment of TNFα-mediated disease or disorders.

### CROSS-REFERENCES TO RELATED APPLICATIONS

This application claims priority to U.S. Provisional Application No. 61/683,681, filed Aug. 15, 2012, the disclosure of which is hereby incorporated by reference in its entirety for all purposes.

### BACKGROUND OF THE INVENTION

Autoimmune diseases, such as Crohn's Disease (CD), ulcerative colitis (UC) and rheumatoid arthritis (RA), are characterized by a dysfunctional immune system in which the overproduction of tumor necrosis factor (TNF-α) is prevalent in the inflamed tissues. The presence of unusually high levels of proinflammatory TNF-α at the sites of inflammation is thought to drive disease pathology, and the removal of excess TNF from sites of inflammation has become a therapeutic goal.

Recombinant monoclonal antibody technology was used to develop the first generation of anti-TNF biologic agents, and in 1998 the US Food and Drug Administration (FDA) approved the use of infliximab (Remicade™) for the treatment of CD (Lee, T. W., & Fedorak, R. N. (2010). Tumor Necrosis Factor-α Monoclonal Antibodies in the Treatment of inflammatory Bowel Disease: Clinical Practice Pharmacology. Gastroenterology Clinics of North America, 39, 543-557). Infliximab is a human-murine chimeric monoclonal antibody comprised of a 25% variable murine Fab′ region linked to the 75% human IgG1:κFc constant region by disulfide bonds (Tracey et al., (2008). Tumor necrosis factor antagonist mechanism of action: A comprehensive review. Pharmacology and Therapeutics, 117, 244-279). Infliximab binds specifically to soluble and membrane-bound TNF-α, preventing it from binding to one of two possible receptors, TNFR1 and TNFR2 (Nesbitt et al. (2009). Certolizumab pegol: a PEGylated anti-tumour necrosis factor alpha biological agent. In F. M. Veronese (Eds.), PEGylated Protein Drugs: Basic Science and Clinical Applications (pp. 229-254). Switzerland: Birkhauser Verlag). As a bivalent mAb, infliximab can bind 2 soluble TNF trimers simultaneously, which allows multimeric complexes to form. Infliximab is known to reduce the levels of TNF-α as well as serum interleukin (IL-6) and acute-phase reactants, such as C-reactive protein (Lee, supra).

In a typical protocol for treating CD patients, infliximab is administered initially as a 5 mg/kg dose at weeks 0, 2, and 6 followed by maintenance doses of 5 mg/kg every 8 weeks. There is a wide fluctuation in serum concentrations of infliximab due to the large intravenous boluses, leading to concentration as high as 100 μg/mL upon injection. The high initial concentration is 13-40 fold greater than the peak concentrations of other TNF antagonists (Tracey et al., supra). Infliximab has a low clearance rate (t_{1/2}=8-10 days) that appears to be independent of typical drug-metabolizing enzymes and is most likely caused by nonspecific proteases. The clinical response is strongly correlated with serum concentrations, and it is likely that antibody formation to infliximab decreases serum levels to non-detectable levels. The variable murine region is thought to be the antigenic component that causes the formation of “antibodies to infliximab” or ATI. Not only does development of ATI lead to increased drug clearance, but it could also result in a range of adverse reactions from mild allergic response to anaphylactic shock. Many patients do not respond to infliximab therapy, and require higher doses or dosing frequency adjustments due to lack of sufficient response (Tracey et al., supra). Furthermore, many patients with secondary response failure to one anti-TNF-α drug benefit from switching to other anti-TNF-α drugs, suggesting a role of neutralizing antibodies.

ELISA assays are currently used to monitor both infliximab and ATI levels in patient serum samples. Typically, the infliximab ELISA utilizes a 96-well microplate ELISA with recombinant TNFα passively adsorbed onto the plate to form the solid phase. The ATI Bridge ELISA employs Infliximab as both capture and detector. While the ELISA assays are robust and sensitive, they have several shortcomings that need to be addressed. Solid phase assays are prone to artifacts such as constraints on the bound antigen that limit its ability to interact with its target, often leading to decreased binding affinity. In the case of the infliximab ELISA assay, this limitation prevents detection of total infliximab in circulation. Only free infliximab can be detected, preventing analysis of patient serum with moderate to high ATI levels. Similarly, only free ATI can be detected in the Bridge ELISA, preventing the detection of total ATI in circulation.

In view of the foregoing, there is a need for new assays to measure anti-TNFα drugs as well as the presence or level of an autoantibody to an anti-TNFα drug. The present invention satisfies these and other needs.

### BRIEF SUMMARY OF THE INVENTION

The present invention provides assays for detecting and measuring the presence or concentration level of an anti-TNFα drug in a sample. The present invention is useful for optimizing therapy and monitoring patients receiving anti-TNFα drugs to detect their presence and serum concentration levels. In addition, assays are provided herein to detect the presence and measure the amount of autoantibodies (e.g., HACA and/or HAHA) against the drug. The present invention also provides methods for selecting therapy, optimizing therapy, and/or reducing toxicity in subjects receiving anti-TNFα drugs for the treatment of TNFα-mediated diseases or disorders (e.g., inflammatory bowel disease, rheumatoid arthritis, and the like).

In one embodiment, the present invention provides a method for determining the presence or level of an anti-TNFα drug in a sample, comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

In another embodiment, the present invention provides a method for determining the presence or level of an autoantibody to an anti-TNFα drug in a sample, comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

In some embodiments, the present invention provides a method to determine the total amount of autoantibody in a sample. This total amount of autoantibody in a sample is the sum of autoantibody bound to unlabeled anti-TNFα drug plus the amount of autoantibody bound to labeled anti-TNFα drug. As such, in one embodiment, the present invention provides a method for determining the total amount of an autoantibody in a sample, comprising:
(a) determining the level of autoantibody bound to labeled anti-TNFα drug according to the following method:
(i) contacting a labeled anti-TNFα drug with the sample to form a labeled complex with the autoantibody;
(ii) subjecting the labeled complex to size exclusion chromatography to separate the labeled complex from free labeled anti-TNFα drug and to measure the amount of the labeled complex and the amount of the free labeled anti-TNFα drug;
(iii) calculating a ratio of the amount of the labeled complex to the sum of the amount of the labeled complex plus free labeled anti-TNFα drug;
(iv) comparing the ratio calculated in step (iii) to a standard curve of known amounts of the autoantibody, to thereby determine the presence or level of the autoantibody, as being the amount of autoantibody bound to a labeled anti-TNFα drug; and
(b) adding the amount of autoantibody bound to unlabeled anti-TNFα drug to the level determined in step (iv) to produce the total amount of autoantibody in the sample.

Accordingly, in some aspects, the methods of the invention provide information useful for guiding treatment decisions for patients receiving or about to receive anti-TNFα drug therapy, e.g., by selecting an appropriate anti-TNFα therapy for initial treatment, by determining when or how to adjust or modify (e.g., increase or decrease) the subsequent dose of an anti-TNFαdrug, by determining when or how to combine an anti-TNFα drug (e.g., at an initial, increased, decreased, or same dose) with one or more immunosuppressive agents such as methotrexate (MTX) and/or azathioprine (AZA), and/or by determining when or how to change the current course of therapy (e.g., switch to a different anti-TNFα drug or to a drug that targets a different mechanism such as an IL-6 receptor-inhibiting monoclonal antibody).

These and other objects, features, and advantages of the present invention will become more apparent when read with the following detailed description and figures which follow.

### BRIEF DESCRIPTION OF THE DRAWINGS

FIG. 1A and FIG. 1B show exemplary embodiments of the assays of the present invention wherein size exclusion HPLC is used to detect binding. FIG. 1A shows a chromatogram of TNFα-Alexa488 and a control. FIG. 1B shows a chromatogram of TNFα-Alexa488 plus infliximab.

FIG. 2 shows an example of a standard curve for an infliximab HPLC mobility shift assay.

FIGS. 3A-3D show exemplary embodiments of the assays of the present invention. FIG. 3A shows a chromatogram of 37.5 ng Infliximab-Alexa488. FIG. 3B shows 37.5 ng Infliximab-Alexa488 plus 1% ATI Positive Serum. FIG. 3C shows a chromatogram of ADL-Alexa488 and a control. FIG. 3D shows a chromatogram of adalimumab-Alexa488 plus ATA.

FIG. 4 shows an example of a standard curve for an autoantibody to infliximab HPLC mobility shift assay.

FIG. 5 shows an example of a standard curve for an autoantibody to adalimumab HPLC mobility shift assay.

### DETAILED DESCRIPTION OF THE INVENTION

### I. Definitions

As used herein, the following terms have the meanings ascribed to them unless specified otherwise.

The terms “anti-TNFα drug” or “TNFα inhibitor” as used herein is intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or normaturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signaling or any other means resulting in decreased TNFα activity in a subject. The term “anti-TNFα drug” or “TNFα inhibitor” preferably includes agents which interfere with TNFα activity. Examples of anti-TNFα drugs include, without limitation, infliximab (REMICADE™, Johnson and Johnson), human anti-TNF monoclonal antibody adalimumab (D2E7/HUMIRA™, Abbott Laboratories), etanercept (ENBREL™, Amgen), certolizumab pegol (CIMZIA®, UCB, Inc.), golimumab (SIMPONI®; CNTO 148), CDP 571 (Celltech), CDP 870 (Celltech), as well as other compounds which inhibit TNFα activity, such that when administered to a subject suffering from or at risk of suffering from a disorder in which TNFα activity is detrimental (e.g., RA), the disorder is treated.

The term “TNFα” is intended to include a human cytokine that exists as a 17 kDa secreted form and a 26 kDa membrane associated form, the biologically active form of which is composed of a trimer of noncovalently bound 17 kDa molecules. The structure of TNFα is described further in, for example, Jones et al., Nature, 338:225-228 (1989). The term TNFα is intended to include human TNFα, a recombinant human TNFα (rhTNF-α), or TNFα that is at least about 80% identity to the human TNFα protein. Human TNFα consists of a 35 amino acid (aa) cytoplasmic domain, a 21 aa transmembrane segment, and a 177 aa extracellular domain (ECD) (Pennica, D. et al. (1984) Nature 312:724). Within the ECD, human TNFα shares 97% aa sequence identity with rhesus TNFα, and 71% to 92% aa sequence identity with bovine, canine, cotton rat, equine, feline, mouse, porcine, and rat TNFα. TNFα can be prepared by standard recombinant expression methods or purchased commercially (R & D Systems, Catalog No. 210-TA, Minneapolis, Minn.).

In certain embodiments, “TNFα” is an “antigen,” which includes a molecule or a portion of the molecule capable of being bound by an anti-TNF-α drug. TNFα can have one or more than one epitope. In certain instances, TNFα will react, in a highly selective manner, with an anti-TNFα antibody. Preferred antigens that bind antibodies, fragments, and regions of anti-TNFα antibodies include at least 5 amino acids of human TNFα. In certain instances, TNFα is a sufficient length having an epitope of TNFα that is capable of binding anti-TNFα antibodies, fragments, and regions thereof.

The term “predicting responsiveness to an anti-TNFα drug” is intended to refer to an ability to assess the likelihood that treatment of a subject with an anti-TNFα drug will or will not be effective in (e.g., provide a measurable benefit to) the subject. In particular, such an ability to assess the likelihood that treatment will or will not be effective typically is exercised after treatment has begun, and an indicator of effectiveness (e.g., an indicator of measurable benefit) has been observed in the subject. Particularly preferred anti-TNFα drugs are biologic agents that have been approved by the FDA for use in humans in the treatment of TNFα-mediated diseases or disorders and include those anti-TNFα drugs described herein.

The term “size exclusion chromatography” or “SEC” includes a chromatographic method in which molecules in solution are separated based on their size and/or hydrodynamic volume. It is applied to large molecules or macromolecular complexes such as proteins and their conjugates. Typically, when an aqueous solution is used to transport the sample through the column, the technique is known as gel filtration chromatography.

The terms “complex,” “immuno-complex,” “conjugate,” and “immunoconjugate” include, but are not limited to, TNFα bound (e.g., by non-covalent means) to an anti-TNFα drug, an anti-TNFα drug bound (e.g., by non-covalent means) to an autoantibody against the anti-TNFα drug, and an anti-TNFα drug bound (e.g., by non-covalent means) to both TNFα and an autoantibody against the anti-TNFα drug.

As used herein, an entity that is modified by the term “labeled” includes any entity, molecule, protein, enzyme, antibody, antibody fragment, cytokine, or related species that is conjugated with another molecule or chemical entity that is empirically detectable. Chemical species suitable as labels for labeled-entities include, but are not limited to, fluorescent dyes, e.g. Alexa Fluor® dyes such as Alexa Fluor® 647, Alexa Fluor® 488, quantum dots, optical dyes, luminescent dyes, and radionuclides, e.g. ¹²⁵I. Additional labels are described in further detail below.

The term “effective amount” includes a dose of a drug that is capable of achieving a therapeutic effect in a subject in need thereof as well as the bioavailable amount of a drug. The term “bioavailable” includes the fraction of an administered dose of a drug that is available for therapeutic activity. For example, an effective amount of a drug useful for treating diseases and disorders in which TNF-α has been implicated in the pathophysiology can be the amount that is capable of preventing or relieving one or more symptoms associated therewith.

The phrase “fluorescence label detection” includes a means for detecting a fluorescent label. Means for detection include, but are not limited to, a spectrometer, a fluorimeter, a photometer, and a detection device commonly incorporated with a chromatography instrument such as, but not limited to, size exclusion-high performance liquid chromatography, such as, but not limited to, an Agilent-1200 HPLC System.

The phrase “optimize therapy” includes optimizing the dose (e.g., the effective amount or level) and/or the type of a particular therapy. For example, optimizing the dose of an anti-TNFα drug includes increasing or decreasing the amount of the anti-TNFα drug subsequently administered to a subject. In certain instances, optimizing the type of an anti-TNFα drug includes changing the administered anti-TNFα drug from one drug to a different drug (e.g., a different anti-TNFα drug). In other instances, optimizing therapy includes co-administering a dose of an anti-TNFα drug (e.g., at an increased, decreased, or same dose as the previous dose) in combination with an immunosuppressive drug.

The term “co-administer” includes to administer more than one active agent, such that the duration of physiological effect of one active agent overlaps with the physiological effect of a second active agent.

The term “subject,” “patient,” or “individual” typically refers to humans, but also to other animals including, e.g., other primates, rodents, canines, felines, equines, ovines, porcines, and the like.

The term “course of therapy” includes any therapeutic approach taken to relieve or prevent one or more symptoms associated with a TNFα-mediated disease or disorder. The term encompasses administering any compound, drug, procedure, and/or regimen useful for improving the health of an individual with a TNFα-mediated disease or disorder and includes any of the therapeutic agents described herein. One skilled in the art will appreciate that either the course of therapy or the dose of the current course of therapy can be changed (e.g., increased or decreased) based upon the presence or concentration level of TNFα, anti-TNFα drug, and/or anti-drug antibody using the methods of the present invention.

The term “immunosuppressive drug” or “immunosuppressive agent” includes any substance capable of producing an immunosuppressive effect, e.g., the prevention or diminution of the immune response, as by irradiation or by administration of drugs such as anti-metabolites, anti-lymphocyte sera, antibodies, etc. Examples of immunosuppressive drugs include, without limitation, thiopurine drugs such as azathioprine (AZA) and metabolites thereof; anti-metabolites such as methotrexate (MTX); sirolimus (rapamycin); temsirolimus; everolimus; tacrolimus (FK-506); FK-778; anti-lymphocyte globulin antibodies, anti-thymocyte globulin antibodies, anti-CD 3 antibodies, anti-CD4 antibodies, and antibody-toxin conjugates; cyclosporine; mycophenolate; mizoribine monophosphate; scoparone; glatiramer acetate; metabolites thereof; pharmaceutically acceptable salts thereof; derivatives thereof; prodrugs thereof; and combinations thereof.

The term “thiopurine drug” includes azathioprine (AZA), 6-mercaptopurine (6-MP), or any metabolite thereof that has therapeutic efficacy and includes, without limitation, 6-thioguanine (6-TG), 6-methylmercaptopurine riboside, 6-thioinosine nucleotides (e.g., 6-thioinosine monophosphate, 6-thioinosine diphosphate, 6-thioinosine triphosphate), 6-thioguanine nucleotides (e.g., 6-thioguanosine monophosphate, 6-thioguanosine diphosphate, 6-thioguanosine triphosphate), 6-thioxanthosine nucleotides (e.g., 6-thioxanthosine monophosphate, 6-thioxanthosine diphosphate, 6-thioxanthosine triphosphate), derivatives thereof, analogues thereof, and combinations thereof.

The term “sample” includes any biological specimen obtained from an individual. Samples include, without limitation, whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample such as a biopsy of a site of inflammation (e.g., needle biopsy), cellular extracts thereof, and an immunoglobulin enriched fraction derived from one or more of these bodily fluids or tissues. In some embodiments, the sample is whole blood, a fractional component thereof such as plasma, serum, or a cell pellet, or an immunoglobulin enriched fraction thereof. One skilled in the art will appreciate that samples such as serum samples can be diluted prior to the analysis. In certain embodiments, the sample is obtained by isolating PBMCs and/or PMN cells using any technique known in the art. In certain other embodiments, the sample is a tissue biopsy such as, e.g., from a site of inflammation such as a portion of the gastrointestinal tract or synovial tissue.

### II. Embodiments

The present invention provides assays for detecting and measuring the presence or level of an anti-TNFα drug and/or the presence or level of autoantibodies to anti-TNFα drugs in a sample. In one aspect, the present invention provides assays for detecting and measuring the presence or level of infliximab (IFX) and/or the presence or level of autoantibodies to infliximab (ATI) in a sample. In another aspect, the present invention provides assays for detecting and measuring the presence or level of adalimumab (ADL) and/or the presence or level of autoantibodies to adalimumab (ATA) in a sample. The present invention is useful for optimizing therapy and monitoring patients receiving anti-TNFα drug therapeutics to detect the presence or level of autoantibodies (e.g., HACA and/or HAHA) against the drug. The present invention also provides methods for selecting therapy, optimizing therapy, and/or reducing toxicity in subjects receiving anti-TNFα drugs for the treatment of TNFα-mediated disease or disorders.

The following applications disclose related technology and are hereby incorporated by reference in their entirety for all purposes: US Patent App. Pub. No. US 2012/329172 and International App. Pub. Nos. WO 2012/054532 and WO 2013/006810.

A. Assay for an Anti-TNFα Drug

In one embodiment, the present invention provides a method for determining the presence or level of an anti-TNFα drug in a sample, comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of the free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

In certain aspects, the assay is performed by incubating fluorescently labeled recombinant TNF-α (e.g., TNF-α Alexa488) and optionally containing a deactivated Alexa488 loading control with a sample such as serum containing infliximab, which is allowed to reach equilibrium, to form various complexes of increasing molecular weight. Complexes are formed ranging in size from approximately 200 kDa for 1:1 binding to over 2000 kDa.

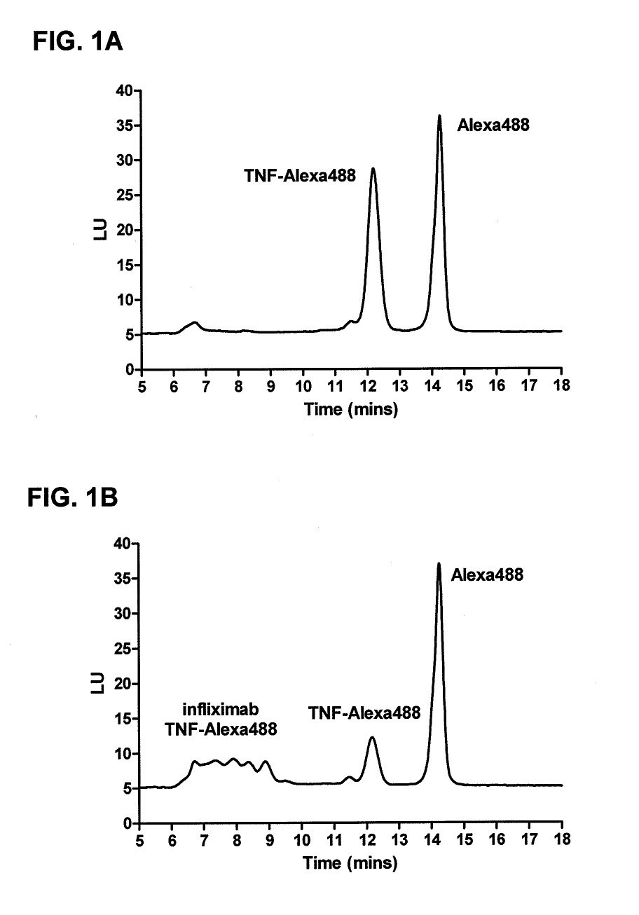

As shown in FIG. 1, after injection and elution of the complex mixture through a column packed with, for example, a gel media, free TNFα-Alexa488 (Mw˜51 kDa) elutes at a retention time (Rₜ) of approximately 11-12.5 minutes (FIG. 1A) while infliximab-TNFα-Alexa488 complexes (FIG. 1B) elute at the range from 6-10 minutes, and the deactivated Alexa488 loading control elutes at about 13.5-14.5 minutes. The assay of the present invention resolves infliximab-TNFα complexes from free TNFα based on the size of the complexes formed. Preferably, the labeled complex is eluted first, followed by the free labeled TNFα.

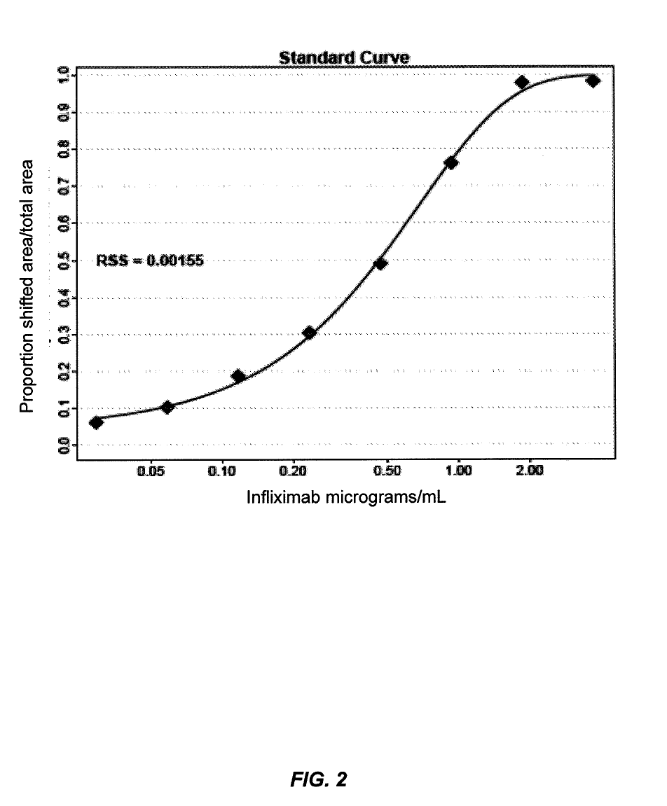

As shown in FIG. 1, quantification can be performed by tracking the appearance of high molecular weight peaks (infliximab-TNFα-Alexa488 complexes (FIG. 1B)) and/or the disappearance of the free labeled TNFα peak (Rₜ=11-12.5 min). FIG. 2 shows an exemplary standard curve generated when the y-axis comprises a ratio, wherein the ratio has a numerator which is labeled complex (e.g., labeled TNFα bound to an anti-TNFα drug) and a denominator which is the sum of the labeled complex plus free labeled TNFα. The x-axis comprises known amounts of the anti-TNFα drug (e.g., IFX, ADL, and the like)

The infliximab standard curve and positive controls are prepared by diluting infliximab in normal human serum. In certain instances, standard samples (e.g., 0.73 to 46.88 μg/mL) and high (e.g., 15.63 μg/mL), medium (e.g., 7.81 μg/mL) and low (e.g., 3.91 μg/mL) infliximab positive controls are run during each assay.

Quantification of the infliximab assay is performed by tracking the appearance of high molecular weight peaks (Rₜ=6-10 min) or the disappearance of the free labeled TNF peak (Rₜ=11-12.5 min). Preferably, raw chromatograms are collected in automated analysis. The fraction of the shifted area representing infliximab-TNF-Alexa488 complexes is plotted from an infliximab standard curve and fitted with a 5-parameter logistic model to account for asymmetry.

In certain instances, the areas under the bound TNFα peak, free TNFα peak and control peak are found by integrating the peak areas. The proportion of the TNFα peak area shifted to bound from free is then calculated for each sample by using the following formula:

p_{D}=b_{D}/(b_{D}±f_{D})

Where p_{D}=proportion of shifted area, b_{D}=area under the bound TNFα-infliximab peak, and f_{D}=the area under the free TNFα peak. Optionally, the ratio of free peak area to control peak area is also calculated. The standard curve has a y-axis of p_{D}, and an x-axis of known amounts of anti-TNFα drug. Using the standard curve, concentrations of control samples, unknown samples, and test samples are interpolated and determined.

Suitable anti-TNFα drugs include, but are not limited to, REMICADE™ (infliximab), ENBREL™ (etanercept), HUMIRA™ (adalimumab), CIMZIA® (certolizumab pegol), and combinations thereof. In one preferred embodiment, the anti-TNFα drug is REMICADE™ (infliximab). In another preferred embodiment, the anti-TNFα drug is CIMZIA® (adalimumab). As a skilled artisan will appreciate, the steps of the foregoing and following methods do not necessarily have to be performed in the particular order in which they are presented.

B. Assay for Autoantibody to Anti-TNFα Drug

In another embodiment, the present invention provides a method for determining the presence or level of an autoantibody to an anti-TNFα drug in a sample, comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

In some embodiments, prior to step (a) the sample is contacted with an acid to dissociate any anti-TNFα drug bound to an autoantibody against the anti-TNFα drug in the sample. In certain instances, the acid comprises an organic acid. In other embodiments, the acid comprises an inorganic acid. In further embodiments, the acid comprises a mixture of an organic acid and an inorganic acid. Non-limiting examples of organic acids include citric acid, isocitric acid, glutamic acid, acetic acid, lactic acid, formic acid, oxalic acid, uric acid, trifluoroacetic acid, benzene sulfonic acid, aminomethanesulfonic acid, camphor-10-sulfonic acid, chloroacetic acid, bromoacetic acid, iodoacetic acid, propanoic acid, butanoic acid, glyceric acid, succinic acid, malic acid, aspartic acid, and combinations thereof. Non-limiting examples of inorganic acids include hydrochloric acid, nitric acid, phosphoric acid, sulfuric acid, boric acid, hydrofluoric acid, hydrobromic acid, and combinations thereof.

In certain embodiments, the amount of an acid corresponds to a concentration of from about 0.01M to about 10M, about 0.1M to about 5M, about 0.1M to about 2M, about 0.2M to about 1M, or about 0.25M to about 0.75M of an acid or a mixture of acids. In other embodiments, the amount of an acid corresponds to a concentration of greater than or equal to about 0.01M, 0.05M, 0.1M, 0.2M, 0.3M, 0.4M, 0.5M, 0.6M, 0.7M, 0.8M, 0.9M, 1M, 2M, 3M, 4M, 5M, 6M, 7M, 8M, 9M, or 10M of an acid or a mixture of acids. The pH of the acid can be, for example, about 0.1, 0.5, 1.0, 1.5, 2.0, 2.5, 3.0, 3.5, 4.0, 4.5, 5.0, 5.5, 6.0, or 6.5.

In some embodiments, the sample is contacted with an acid an amount of time that is sufficient to dissociate preformed complexes of the autoantibody and the anti-TNFα drug. In certain instances, the sample is contacted (e.g., incubated) with an acid for a period of time ranging from about 0.1 hours to about 24 hours, about 0.2 hours to about 16 hours, about 0.5 hours to about 10 hours, about 0.5 hours to about 5 hours, or about 0.5 hours to about 2 hours. In other instances, the sample is contacted (e.g., incubated) with an acid for a period of time that is greater than or equal to about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10 hours. The sample can be contacted with an acid at 4° C., room temperature (RT), or 37° C. In one embodiment, the acid is 0.5M Citric Acid pH 3.0 for one hour.

In some embodiments, the sample after acid dissociation treatment is neutralized to raise the pH with a buffer, such as PBS. In some embodiments, the sample after acid dissociation treatment is contacted with a buffer such that the sample is in an environment suitable for immune complexes to form between fluorescent-labeled anti-TNF

An illustrative description of a method for detecting and measuring the presence or level of infliximab (IFX) and/or the presence or level of autoantibodies to infliximab (ATI) in a sample is present below.

In certain aspects, the method includes a first step of acid dissociating any infliximab (IFX) bound to an autoantibody against infliximab present in the standards, controls and samples. For instances, an acid is contacted with the sample for an incubation period (e.g., room temperature for one hour). Labeled IFX (e.g., fluorescently labeled IFX such as IFX-Alexa488) and optionally, a deactivated Alexa488 loading control, is then added to in excess to compete with free IFX in the samples. The reaction is allowed to reach equilibrium.

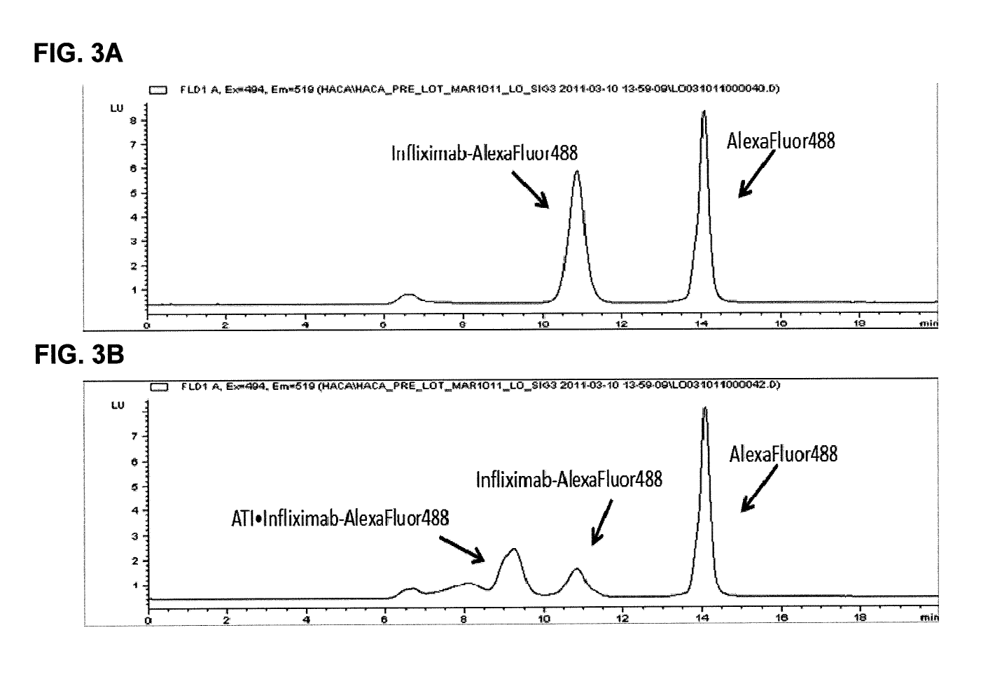

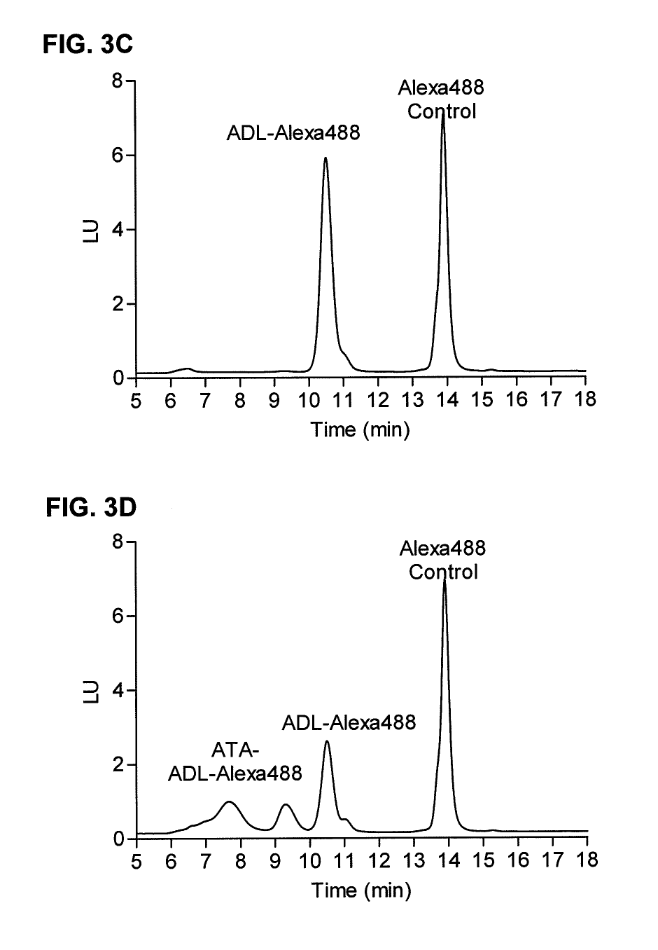

Turning now to FIG. 3A-B, complexes are formed and range in size from approximately 300 kDa for 1:1 binding to over 2000 kDa. Prior to injection, all reaction solutions (e.g., samples, standards and controls) are diluted and filtered through a filter plate. After injection and elution of the complex mixture through a column packed with gel media, free labeled anti-TNFα drug (e.g., infliximab-Alexa488 (Mw˜150 kDa, FIG. 3A)) elutes at a retention time of approximately 10-11.5 minutes while the complexes of anti-TNFα drug bound to an autoantibody against the anti-TNFα drug (e.g., ATI-Infliximab-Alexa488 complexes, FIG. 3B)) elute at the range from 6-10 minutes, and the deactivated control (e.g., Alexa488 loading control) elutes between 13.5-14.5 minutes. This real time, liquid phase assay resolves an anti-TNFα drug bound to an autoantibody against the anti-TNFα drug (e.g., ATI-Infliximab complexes) from free anti-TNFα drug (e.g., infliximab) based on the size of the complexes formed.

In some embodiments, the method of the present invention for determining the presence or level of an autoantibody to an anti-TNFα drug in a sample is performed in an automated mode. For example, in one embodiment, the automated assay comprises an automated liquid handler and an HPLC system. In some instances, the reagents, samples and other fluid components of the assay are transferred using an automated liquid handling robot, including, but not limited to, the Tecan Freedom EVO with TE-VACs, Gilson 215 or Agilent Bravo. Non-limiting examples of an HPLC system are available from Agilent Technologies (Santa Clara, Calif.), Shimadzu (Pleasanton, Calif.), and Dionex Corp. (Sunnyvale, Calif.). In some embodiments, size exclusion chromatography is performed using a gel filtration column such as aPhenomenx BioSep SEC-S3000 column or any column with a substantially similar size exclusion range.

In one embodiment, the ATI assay is performed by first acid dissociating Infliximab-ATI complexes in the standards, controls and sample. Fluorescently labeled infliximab (infliximab-Alexa488) containing an optional deactivated Alexa488 loading control is then added in excess to compete with free infliximab in the sample. A buffer is used to neutralize the reactions and all reactions are incubated for one hour to achieve equilibrium, forming various complexes of increasing molecular weight. Complexes formed range in size from approximately 300 kDa for 1:1 binding to over 2000 kDa. Prior to injection, all reaction solutions are diluted (e.g., with human serum, animal serum or BSA) and filtered through a filter plate (e.g., 0.22 μM filter plate). After injection and elution of the complex mixture through a column packed with gel media, free Infliximab-Alexa488 (M_{w}˜150 kDa) elutes at a retention time of approximately 10-11.5 minutes while ATI-Infliximab-Alexa488 complexes elute at the range from 6-10 minutes, and the optional deactivated Alexa488 loading control elutes between 13.5-14.5 minutes.

Standards and control samples for detecting ATI include, without limitation, pooled ATI positive human serum and any rabbit polyclonal antibody (e.g., whole antibody and F(ab′)2 fragment) that binds to infliximab. In some embodiments, the standards and control samples also include a diluent such as, but not limited to, normal human serum, normal rabbit serum, or BSA.

In some embodiments, a standard curve (e.g., 1.56 to 200 U/mL or 3.125 to 200 U/mL) and high (e.g., 100 U/mL or 80 U/mL), med (e.g., 50 U/mL or 20 U/mL) and low (e.g., 25 or 5 U/mL) U/mL) ATI positive controls are run during each assay.

The level of ATI is determined by the ratio of the shifted area to the free IFX peak and normalized to the internal control. Quantification of the infliximab and ATI are performed by tracking the appearance of high molecular weight peaks (Rt=6-10) and the disappearance of the free infliximab peak (Rt=10-11.5). Raw chromatograms are collected and undergo statistical analysis. The analysis includes normalizing the spectra, finding the areas under each peak, and calculating the proportion of peak area shifted to bound TNF-infliximab as a function of the total TNF/infliximab area (infliximab assay) or the proportion of peak area shifted to bound Infliximab/ATI as a function of the total infliximab/ATI area (ATI assay). With these data, standard curves are made and sample concentrations of infliximab and ATI interpolated.

The ratio of the area representing the free infliximab/Alexa488 loading control is plotted from an ATI standard curve and fit with a 5-parameter logistic model to account for asymmetry. Unknowns are calculated from a standard curve. Concentrations of ATI are reported in U/mL, wherein 100% ATI positive control serum has a concentration of 200 U/mL.

As shown in FIG. 3, quantification is performed by tracking the appearance of high molecular weight peaks (Rₜ=6-10, FIG. 3B) and/or the disappearance of the free Infliximab peak (Rₜ=10-11.5, FIG. 3A).

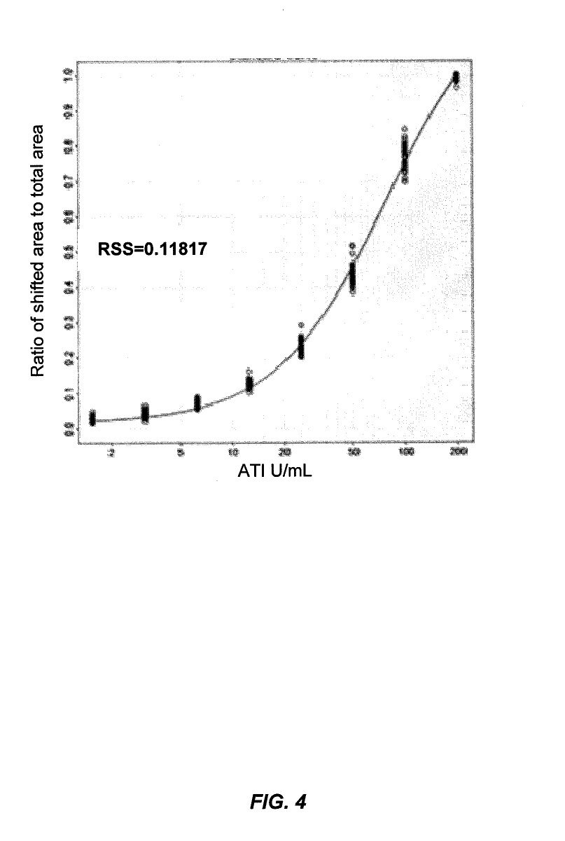

FIG. 4 shows a standard curve generated having a y-axis comprising a ratio, wherein the ratio has a numerator which is the amount of labeled complex (e.g., an anti-TNFα drug bound to an autoantibody against the anti-TNFα drug) and a denominator which is the sum of the amount of the labeled complex plus free labeled anti-TNFα drug. The x-axis comprises known amounts of the autoantibody.

The present invention also provides a method for detecting and measuring the presence or level of adalimumab (ADL) and/or the presence or level of autoantibodies to adalimumab (ATA) in a sample.

In some embodiments, the assay is performed by acid dissociation of the serum proteins in samples collected from patients treated with ADL, followed by addition of fluorescently labeled adalimumab (e.g., ADL-Alexa488) and optionally, a deactivated loading control (e.g., Alexa488). The samples are then neutralized and allowed to reach equilibrium at room temperature to form various immune complexes of increasing molecular weight. The complexes formed range in size from approximately 300 kDa for 1:1 antigen/antibody binding to over 2,000 kDa for multiple antigens/antibodies. After injection and elution of the complex mixture through a column packed with gel media (e.g., Phenomenex BioSep SEC-S3000), free ADL-Alexa488 (Mw˜150 kDa) elutes at a retention time of approximately 10-11.5 minutes while ATA-ADL-Alexa488 complexes elute at the range from 6-10 minutes and the optional deactivated Alexa488 loading control elutes between 13.5-14.5 minutes (FIG. 3C-D). The mobility shift assay of the present invention resolves ATA-adalimumab complexes from free ADL-Alexa488 based on the size of the complexes formed.

Standards and control samples for detecting ATA include, without limitation, pooled ATA positive human serum and any rabbit polyclonal antibody (e.g., whole antibody and F(ab′)2 fragment) that binds to adalimumab. In some embodiments, the standards and control samples also include a diluent such as, but not limited to, normal human serum, normal rabbit serum, or BSA.

In some embodiments, a series of standard samples are prepared by about 2-fold serial dilutions. In some instances, the standard sample that generates a complete shift for the first standard curve point and then a partial shift for the second is assigned the value of 200 U/mL.

The level of ATA is determined by the ratio of the shifted area to the free ADL peak and normalized to the internal control. Quantification can be performed by tracking the appearance of the high molecular weight peaks (Rₜ=6-10 min) or the disappearance of the free ADL-Alexa488 peak (Rₜ=10-11.5 min). Product appearance and substrate disappearance are linked by the stoichiometry of the reaction, enabling the measurement either or both concentrations. Raw chromatograms are collected and undergo statistical analysis. In some embodiments, fractions of the shifted area representing ATA-ADL-Alexa488 complexes from different concentrations of added ATA are used to generate an ATA standard curve and fitted with a 5-parameter logistic (5-PL) model to account for asymmetry.

In some embodiments, analysis of the raw chromatograms includes normalizing the data with respect to retention time by forcing the Alexa488 control peak of each spectrum to be a set time (e.g., 14 minutes). In some instances, the spectrum baseline (x-axis) of the chromatogram can be normalized in the following steps: 1) subtracting from each data point in each spectrum the luminescent unit (LU) value from the background serum sample; and 2) creating a linear model to describe the baseline using two data points at the 10^{th} and 90^{th} percentile retention times such that the baseline is as flat and as close to zero luminescent units (LU) as possible.

In some instances, a peak detection algorithm is used to find all the peaks and troughs in each spectrum per assay. In one embodiment, a cubic smoothing spline is fit to each spectrum, and peaks and troughs are defined as a change in the first derivative of the signal. A peak is a sign change of the spectrum's slope from positive to negative. Conversely, troughs are defined as a change in sign from negative to positive. For instance, the tallest peak within a window at the expected location of the free ADL-Alexa488 peak (e.g., 10 to 11 minutes) is taken to be a free peak itself. The troughs directly above and below the detected free peak define the upper and lower limits of the peak itself. In some embodiments, the bound area is comprised of several different autoantibody to anti-TNFα drug-anti-TNFα drug complexes of varying stoichiometry, such that its upper limit is defined as the lower limit of the free peak, and the bound peaks' lower limit is arbitrarily set at a low, but adjustable, retention time (e.g, about 5 minutes).

In certain instances, the areas under the bound anti-TNFα drug peak, free anti-TNFα drug peak and control peak are found by integrating the peak areas. The proportion of the anti-TNFα drug peak area shifted to bound from free is then calculated for each sample by using the formula:

p_{A}=b_{A}/(b_{A}+f_{A})

Where p_{A}=proportion of shifted area, b_{A}=area under the bound anti-TNFα drug peak, and f_{A}=the area under the free anti-TNFα drug. Optionally, the ratio of free peak area to control peak area is also calculated. The standard curve has a y-axis of p_{A}, and an x-axis of known amounts of autoantibodies against the anti-TNFα drug.

In particular embodiments, the sample is contacted with an amount of an acid that is sufficient to dissociate preformed complexes of the autoantibody and the anti-TNFα drug, such that the labeled anti-TNFα drug, the unlabeled anti-TNFα drug, and the autoantibody to the anti-TNFα drug can equilibrate and form complexes therebetween.

In preferred embodiments, the methods of the invention comprise detecting the presence or level of the autoantibody without substantial interference from the anti-TNFα drug that is also present in the sample. In such embodiments, the sample can be contacted with an amount of an acid that is sufficient to allow for the detection and/or measurement of the autoantibody in the presence of a high level of the anti-TNFα drug. In some embodiments, the phrase “high level of an anti-TNFα drug” includes drug levels of from about 10 to about 100 μg/mL, about 20 to about 80 μg/mL, about 30 to about 70 μg/mL, or about 40 to about 80 μg/mL. In other embodiments, the phrase “high level of an anti-TNFα drug” includes drug levels greater than or equal to about 10, 20, 30, 40, 50, 60, 70, 80, 90, or 100 μg/mL.

C. Total Amount of Autoantibody Against the Anti-TNFα Drug

In some embodiments, the present invention provides a method to determine the total amount of autoantibody against the anti-TNFα drug in a sample. This total amount of autoantibody is the sum of autoantibody bound to unlabeled anti-TNFα drug plus the amount of autoantibody bound to labeled anti-TNFα drug. In certain instances, the autoantibody assays are performed by first acid dissociating anti-TNFα drug-autoantibody complexes in the standards, controls, samples, or a combination thereof.

As such, in one embodiment, the present invention provides a method comprising:
(a) determining the level of autoantibody bound to labeled anti-TNFα drug according to the following method:
   (i) contacting a labeled anti-TNFα drug with the sample to form a labeled complex with the autoantibody;
   (ii) subjecting the labeled complex to size exclusion chromatography to separate the labeled complex from free labeled anti-TNFα drug and to measure the amount of the labeled complex and the amount of the free labeled anti-TNFα drug;
   (iii) calculating a ratio of the amount of the labeled complex to the sum of the amount of the labeled complex plus free labeled anti-TNFα drug; and
   (iv) comparing the ratio calculated in step (iii) to a standard curve of known amounts of the autoantibody, to thereby determine the presence or level of the autoantibody, as being the amount of autoantibody bound to a labeled anti-TNFα drug; and
(b) adding the amount of autoantibody bound to unlabeled anti-TNFα drug to the level determined in step (iv) to produce the total amount of autoantibody in the sample.

In one aspect, the amount of autoantibody bound to unlabeled anti-TNFα drug is calculated by multiplying the level of autoantibody bound to labeled anti-TNFα drug of step (iv) by the amount of unlabeled anti-TNFα drug divided by the amount of labeled anti-TNFα drug.

In some aspects, the amount of unlabeled anti-TNFα drug is the weight of anti-TNFα drug. This weight can be determined by multiplying the concentration of anti-TNFα drug by the volume of serum in the assay to determine the amount of autoantibody bound to labeled anti-TNFα drug.

In some aspects, the amount of labeled anti-TNFα drug is the weight of labeled anti-TNFα drug determined by multiplying the volume of labeled anti-TNFα drug by the concentration of labeled anti-TNFα drug added to the sample.

D. Labels

An anti-TNFα drug and/or TNFα can be labeled with any of a variety of one or more detectable group(s). In preferred embodiments, an anti-TNFα drug and/or TNFα is labeled with a fluorophore or a fluorescent dye. Non-limiting examples of fluorophores or fluorescent dyes include those listed in the Molecular Probes Catalogue, which is herein incorporated by reference (see, R. Haugland, The Handbook—A Guide to Fluorescent Probes and Labeling Technologies, 10^{th} Edition, Molecular probes, Inc. (2005)). Such exemplary fluorophores or fluorescent dyes include, but are not limited to, Alexa Fluor® dyes such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, and/or Alexa Fluor® 790, as well as other fluorophores including, but not limited to, Dansyl Chloride (DNS-Cl), 5-(iodoacetamida)fluoroscein (5-IAF), fluoroscein 5-isothiocyanate (FITC), tetramethylrhodamine 5-(and 6-)isothiocyanate (TRITC), 6-acryloyl-2-dimethylaminonaphthalene (acrylodan), 7-nitrobenzo-2-oxa-1,3,-diazol-4-yl chloride (NBD-Cl), ethidium bromide, Lucifer Yellow, 5-carboxyrhodamine 6G hydrochloride, Lissamine rhodamine B sulfonyl chloride, Texas Red™ sulfonyl chloride, BODIPY™, naphthalamine sulfonic acids (e.g., 1-anilinonaphthalene-8-sulfonic acid (ANS), 6-(p-toluidinyl)naphthalen-e-2-sulfonic acid (TNS), and the like), Anthroyl fatty acid, DPH, Parinaric acid, TMA-DPH, Fluorenyl fatty acid, fluorescein-phosphatidylethanolamine, Texas Red-phosphatidylethanolamine, Pyrenyl-phophatidylcholine, Fluorenyl-phosphotidylcholine, Merocyanine 540, 1-(3-sulfonatopropyl)-4[β-[2[(di-n-butylamino)-6 naphthyl]vinyl]pyridinium betaine (Naphtyl Styryl), 3,3′ dipropylthiadicarbocyanine (diS-C₃-(5)), 4-(p-dipentyl aminostyryl)-1-methylpyridinium (di-5-ASP), Cy-3 Iodo Acetamide, Cy-5-N-Hydroxysuccinimide, Cy-7-Isothiocyanate, rhodamine 800, IR-125, Thiazole Orange, Azure B, Nile Blue, Al Phthalocyanine, Oxaxine 1,4′,6-diamidino-2-phenylindole (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARE-6, BAPTA, coumarin, phytofluors, Coronene, metal-ligand complexes, IRDye® 700DX, IRDye® 700, IRDye® 800RS, IRDye® 800CW, IRDye® 800, Cy5, Cy5.5, Cy7, DY676, DY680, DY682, DY780, and mixtures thereof. Additional suitable fluorophores include enzyme-cofactors; lanthanide, green fluorescent protein, yellow fluorescent protein, red fluorescent protein, or mutants and derivates thereof. In one embodiment of the invention, the second member of the specific binding pair has a detectable group attached thereto.

Typically, the fluorescent group is a fluorophore selected from the category of dyes comprising polymethines, pthalocyanines, cyanines, xanthenes, fluorenes, rhodamines, coumarins, fluoresceins and BODIPY™.

In one embodiment, the fluorescent group is a near-infrared (NIR) fluorophore that emits in the range of between about 650 to about 900 nm. Use of near infrared fluorescence technology is advantageous in biological assays as it substantially eliminates or reduces background from auto fluorescence of biosubstrates. Another benefit to the near-IR fluorescent technology is that the scattered light from the excitation source is greatly reduced since the scattering intensity is proportional to the inverse fourth power of the wavelength. Low background fluorescence and low scattering result in a high signal to noise ratio, which is essential for highly sensitive detection. Furthermore, the optically transparent window in the near-IR region (650 nm to 900 nm) in biological tissue makes NIR fluorescence a valuable technology for in vivo imaging and subcellular detection applications that require the transmission of light through biological components. Within aspects of this embodiment, the fluorescent group is preferably selected form the group consisting of IRDye® 700DX, IRDye® 700, IRDye® 800RS, IRDye® 800CW, IRDye® 800, Alexa Fluor® 660, Alexa Fluor® 680, Alexa Fluor® 700, Alexa Fluor® 750, Alexa Fluor® 790, Cy5, Cy5.5, Cy7, DY676, DY680, DY682, and DY780. In certain embodiments, the near infrared group is IRDye® 800CW, IRDye® 800, IRDye® 700DX, IRDye® 700, or Dynomic DY676.

Fluorescent labeling is accomplished using a chemically reactive derivative of a fluorophore. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide, many of which are commercially available. Reaction of any of these reactive dyes with an anti-TNFα drug results in a stable covalent bond formed between a fluorophore and an anti-TNFα drug.

In certain instances, following a fluorescent labeling reaction, it is often necessary to remove any nonreacted fluorophore from the labeled target molecule. This is often accomplished by size exclusion chromatography, taking advantage of the size difference between fluorophore and labeled protein.

Reactive fluorescent dyes are available from many sources. They can be obtained with different reactive groups for attachment to various functional groups within the target molecule. They are also available in labeling kits that contain all the components to carry out a labeling reaction. In one preferred aspect, Alexa Fluor® 647 C2 maleimide is used from Invitrogen (Cat. No. A-20347).

Specific immunological binding of an anti-drug antibody (ADA) to an anti-TNFα drug can be detected directly or indirectly. Direct labels include fluorescent or luminescent tags, metals, dyes, radionuclides, and the like, attached to the antibody. In certain instances, an anti-TNFα drug that is labeled with iodine-125 (¹²⁵I) can be used for determining the concentration levels of ADA in a sample. In other instances, a chemiluminescence assay using a chemiluminescent anti-TNFα drug that is specific for ADA in a sample is suitable for sensitive, non-radioactive detection of ADA concentration levels. In particular instances, an anti-TNFα drug that is labeled with a fluorochrome is also suitable for determining the concentration levels of ADA in a sample. Examples of fluorochromes include, without limitation, Alexa Fluor® dyes, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)₂ anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat F(ab′)₂ anti-human IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of ADA levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

In certain embodiments, size exclusion chromatography is used. The underlying principle of SEC is that particles of different sizes will elute (filter) through a stationary phase at different rates. This results in the separation of a solution of particles based on size. Provided that all the particles are loaded simultaneously or near simultaneously, particles of the same size elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

In certain aspects, the eluent is collected in constant volumes, or fractions. The more similar the particles are in size, the more likely they will be in the same fraction and not detected separately. Preferably, the collected fractions are examined by spectroscopic techniques to determine the concentration of the particles eluted. Typically, the spectroscopy detection techniques useful in the present invention include, but are not limited to, fluorometry, refractive index (RI), and ultraviolet (UV). In certain instances, the elution volume decreases roughly linearly with the logarithm of the molecular hydrodynamic volume (i.e., heaver moieties come off first).

The present invention further provides a kit for detecting the presence or level of an autoantibody to an anti-TNFα drug in a sample. In particular embodiments, the kit comprises one or more of the following components: an acid (or mixture of acids), a labeled anti-TNFα drug (e.g., a labeled anti-TNFα antibody), a labeled internal control, a neutralizing agent (or mixtures thereof), means for detection (e.g., a fluorescence detector), a size exclusion-high performance liquid chromatography (SE-HPLC) instrument, and/or instructions for using the kit.

### III. EXAMPLES

### Example 1

### Mobility Shift Assay for Anti-TNF-α Drug Infliximab

This example illustrates one embodiment of the method described herein for determining the presence of infliximab in a sample. The assay is performed by incubating fluorescently labeled recombinant TNF-α (TNF-Alexa488) containing a deactivated Alexa488 loading control with sera containing infliximab and allowed to reach equilibrium, forming various complexes of increasing molecular weight. Complexes are formed ranging in size from approximately 200 kDa for 1:1 binding to over 2000 kDa. After injection and elution of the complex mixture through a column packed with gel media, free TNF-Alexa488 (Mw˜51 kDa) elutes at a retention time of approximately 11-12.5 minutes while infliximab-TNF-Alexa488 complexes elute at the range from 6-10 minutes, and the deactivated Alexa488 loading control elutes between 13.5-14.5 minutes. This real time, liquid phase assay resolves infliximab-TNF complexes from free TNF based on the size of the complexes formed.

Quantification can be performed by tracking the appearance of high molecular weight peaks (Rₜ=6-10 min) or the disappearance of the free labeled TNF peak (Rₜ=11-12.5 min). FIG. 2 shows an exemplary standard curve. The y-axis comprises a ratio, wherein the ratio has a numerator which is labeled complex (e.g., labeled TNFα bound to an anti-TNFα drug) and a denominator which is the sum of the labeled complex plus free labeled TNFα. The x-axis comprises known amounts of the anti-TNFα drug. Unknowns are determined from the standard curve and the effective concentration of infliximab in 100% serum calculated by multiplying the result by the dilution factor.

The infliximab standard curve and positive controls were prepared by diluting Infliximab in normal human serum. The standard curve (0.73 to 46.88 μg/mL) and High (15.63 μg/mL), Medium (7.81 μg/mL) and Low (3.91 μg/mL) Infliximab Positive Controls (IPC) were run during each assay. The reference range of the assay was less than 1.0 μg/mL. The reportable range was 1.0-34.0 μg/mL. Sample values greater than 34.0 μg/mL were reported as >34.0 μg/mL. Sample values lower than μg/mL were reported as <1.0 μg/mL.

Data analysis is done in an automated manner using a computer program. The program normalizes the spectra, finds the areas under each peak, and calculates the proportion of peak area shifted to bound TNF-infliximab as a function of the total TNF/Infliximab area. With these data, standard curves are made and sample concentrations of Infliximab interpolated.

### Example 2

### Mobility Shift Assay for Autoantibodies Against Anti-TNF-α Drug Infliximab (ATI)

This example illustrates one embodiment of the method described herein for determining the total amount of autoantibody against infliximab present in a sample. The assay was performed by first acid dissociating infliximab-ATI complexes in the standards, controls and patient serum samples with 0.5M Citric Acid pH 3.0 with an one hour incubation. Fluorescently labeled infliximab (infliximab-Alexa488) containing a deactivated Alexa488 loading control was then added in excess to compete with free Infliximab in the samples. 10×PBS was used to neutralize the reactions and all reactions were incubated for one hour to achieve equilibrium, forming various complexes of increasing molecular weight. Complexes formed range in size from approximately 300 kDa for 1:1 binding to over 2000 kDa. Prior to injection, all reaction solutions were diluted to 2% serum and filtered through a 0.22 μM filter plate. After injection and elution of the complex mixture through a column packed with gel media, free Infliximab-Alexa488 (M_{w}˜150 kDa) eluted at a retention time of approximately 10-11.5 minutes while ATI-Infliximab-Alexa488 complexes eluted at the range from 6-10 minutes, and the deactivated Alexa488 loading control eluted between 13.5-14.5 minutes. The method described herein resolved ATI-Infliximab complexes from free Infliximab based on the size of the complexes formed.

Quantification was performed by tracking the appearance of high molecular weight peaks (Rₜ=6-10) and the disappearance of the free Infliximab peak (Rₜ=10-11.5). FIG. 4 shows an exemplary standard curve. The y-axis comprises a ratio, wherein the ratio has a numerator which is the amount of labeled complex (e.g., an anti-TNFα drug bound to an autoantibody against the anti-TNFα drug) and a denominator which is the sum of the amount of the labeled complex plus free labeled anti-TNFα drug. The x-axis comprises known amounts of the autoantibody. Unknowns are calculated from the standard curve. Concentrations of ATI are reported in arbitrary U/mL, 100% ATI Positive Control serum has a concentration of 200 U/mL.

The Residual Sum of Squares (RSS) of the standard curve was determined to judge the quality of the fit. If the RSS was >0.01 (e.g., representing a poor fit), the starting parameters were loosened and a fit was attempted again. If the RSS was still >0.01, the standard with the lowest shifted area was removed, and the statistical analysis were repeated once if RSS>0.01. If the curve adaptation fails]ed once more, wherein RSS>0.01, then the analysis was aborted.

The ATI standard curve and positive controls were prepared by diluting pooled positive serum in normal human serum. The standard curve (1.56 to 100 U/mL) and High (100 U/mL, Med (50 U/mL) and Low (25 U/mL) ATI Positive Controls (APC) were run during each assay. The reference range of the assay was less than 3.1 U/mL. The reportable range was 3.1-100 U/mL. Sample values greater than 100 U/mL were reported as >100 U/mL. Sample values lower than 3.1 U/mL were reported as <3.1 U/mL.

Data analysis was performed in an automated manner using the statistically analysis program R. The analysis normalized the spectra, found the areas under each peak, and calculated the proportion of peak area shifted to bound Infliximab/ATI as a function of the total Infliximab/ATI area. With these data, standard curves were made and sample concentrations of ATI interpolated.

### Example 3

### Calculation of Total Amount of Autoantibody to Infliximab (Total ATI)

This example describes methods of calculating the total amount of autoantibody against infliximab in a sample from a patient.

In this illustrative example, in order to calculate the amount of total autoantibody, the following equation is used:

Total ATI=ATI bound to unlabeled IFX+ATI bound to labeled IFX

### (a) Calculation of ATI Bound to Unlabeled Infliximab

Using the equilibrium equation A+B+C=AC+BC, where A=unlabeled Infliximab, B=Labeled-infliximab and C=ATI, the total amount of ATI present in the serum can be accurately calculated.

For this equation the following values are known for each sample:

A is the concentration calculated from testing with the infliximab mobility shift assay.

B is the known amount of infliximab-AlexaFluor488 spiked into the sample.

BC is the concentration calculated from the ATI mobility shift assay.

Knowing that the sample is acid dissociated and then allowed to reach equilibrium:

$$\frac{BC}{B} = \frac{A C}{A}$$

By solving for AC, the concentration of ATI bound to unlabeled infliximab is obtained. The total ATI in the sample then is equal to AC+BC.

$$ATI bound to unlabeled IFX = \frac{U \text{/} mL ATI from mobility shift assay \times mg unlabeled IFX}{mg labeled IFX}$$

The detailed equation for calculation of ATI bound to unlabeled IFX is as follows:

$$ATI bound to unlabeled IFX = \frac{\begin{matrix}\begin{matrix}U \text{/} mL ATI from mobility shift assay \times \\ μg \text{/} mL IFX in 100 \% Serum \times \end{matrix} \\ Vol of 100 \% serum added to ATI Assay\end{matrix}}{\begin{matrix}Volume of labeled IFX added to sample \times \\ Concentration of labeled IFX added to sample\end{matrix}}$$

### (b) Calculation of Total ATI in Patient Samples

The total concentration of ATI in a patient sample is calculated in the following manner:
The amount of ATI bound to labeled IFX is determined from the ATI mobility shift assay.
If the measurement of ATI bound to labeled IFX is between the limits of quantification, the ATI mobility shift assay result is added to the calculated concentration of antibody bound to intrinsic (unlabeled) IFX to produce the Total ATI concentration.

Total ATI=ATI bound to unlabeled IFX+ATI bound to labeled IFX
The concentration of ATI bound to unlabeled IFX is calculated by multiplying the concentration of ATI bound to labeled IFX by the measured concentration of serum IFX and by the inverse of the concentration of labeled IFX of the sample used to measure concentration of ATI bound to labeled IFX.

$$ATI bound to unlabeled IFX = \frac{\begin{matrix}U \text{/} mL ATI from mobility shift assay \times \\ μg unlabeled IFX\end{matrix}}{μg labeled IFX}$$

### (c) Exemplary Calculation of Total ATI

Example of the calculation for a patient sample with the following mobility shift assay results:

$$ATI = 25 U \text{/} mL$$

$$Infliximab = 1 μg \text{/} mL ( 0.001 mg \text{/} mL in the equation below )$$

$$ATI bound to unlabeled IFX = \frac{\begin{matrix}25 U \text{/} mL ATI \times 0.001 mg \text{/} mL IFX \times \\ 0.024 mL serum in ATI assay\end{matrix}}{0.033 mL of labeled IFX \times 0.0135 mg \text{/} mL of labeled IFX}$$

$$ATI bound to Unlabeled IFX = 1.3 U \text{/} mL$$

$$Total ATI = 25 U \text{/} mL + 1.3 U \text{/} mL$$

$$Total ATI = 26.3 U \text{/} mL$$

(d) Calculation of Total Amount of Autoantibodies (Total ATI) from Automated Mobility Shift Assay

Total ATI was calculated by the following equations:

Partial ATI (U/mL)=ATI Assay Result; wherein the level of ATI in the sample determined by the mobility shift assay as described herein, e.g., Example 2.

Unbound ATI (U/mL)=(IFX Assay Result)×(ATI Assay Result)×(0.05387); wherein IFX Assay Result represents the level of IFX in a sample determined by the mobility shift assay as described herein, e.g., Example 1.

Total ATI (U/mL)=Partial ATI (U/mL)+Unbound ATI

### (e) Exemplary Calculation of Total ATI Automated Mobility Shift Assay

Example of the calculation for a patient sample with the following mobility shift assay results:

ATI=25 U/mL

Infliximab=1 μg/mL

Unbound ATI=1×25×0.05387=1.34675 U/mL

Total ATI=26.34675 U/mL

### Example 4

### Automated Mobility Shift Assays for Infliximab and ATI

This example shows that automated mobility shift assays for infliximab and total ATI, as described herein, can be used an alternative to manual assays described herein. Thus, automated assays can be used for infliximab and total ATI determination.

Regression plots were prepared from the mean values for manual vs. automated assay results. Linear regression analysis of manual vs. automated means for both infliximab and total ATI assays showed slopes of 1.086 and 0.9655, respectively. The r² values 0.9796 and 0.9913 meet the acceptance criteria of r²>0.95. These results demonstrate acceptable response ranges between the two assay formats.

Individual automated assay duplicate values were plotted against the mean manual assay value for each sample. Linear regression analysis of manual means vs. automated duplicates for both infliximab and total ATI assays showed slopes of 1.077 and 0.9658, respectively. The r² values were 0.9703 and 0.9897, respectively. These results also demonstrate acceptable response ranges between the two assay formats.

For an analysis of bias, the difference between the mean manual and automated assay values were plotted against the average of the mean manual and automated assay values for each sample. The horizontal centerline of this plot has the value of zero. Plots for the infliximab and the total ATI assays are produced and summarized in the following table.

| | IFX Difference | Total ATI Difference |
|---|---|---|
| | vs. Average | vs. Average |
| | (Bland-Altman Analysis) | (Bland-Altman Analysis) |
| Bias | 0.29 | −2.9 |
| SD of bias | 1.2 | 4.7 |
| 95% Limits of | | |
| Agreement | | |
| From | −2.1 | −12 |
| To | 2.7 | 6.3 |

In a comparison of the manual and automated infliximab assay values, the bias was 0.29±1.2. The standard deviation (SD) of the bias was used to calculate the limits of agreement. 95% of assay values were predicted to fall between the upper and lower limits of agreement 2.7 and −2.1, respectively. In a comparison of the manual and automated total ATI assay values, the bias was −2.9±4.7.95% of values were predicted to fall between the upper and lower limits of agreement 6.3 and −12, respectively. Analysis revealed that the bias for the manual Infliximab and ATI assays meets the acceptance criteria of ±15%.

In summary. automated mobility shift assays for infliximab and ATI met the acceptance criteria for equivalence to the manual assays for infliximab and ATI, respectively. The automated assays as described herein are an acceptable methods for infliximab and Total ATI determination.

### Example 5

### Mobility Shift Assay for Autoantibodies Against ADL (ATA)

This example illustrates one embodiment of the method described herein for measuring the total amount of ATA present in a sample. First, acid dissociation of the serum proteins was performed on a sample collected from a patient treated with Adalimumab. The sample was contacted with citric acid, pH 3.0 and incubated for one hour at room temperature to free the ATA in the sample from other bound proteins. Next, the sample was contacted with fluorescently labeled Adalimumab (ADL-Alexa488) and a deactivated Alexa488 loading control, and the pH of the sample was neutralized with a 10×PBS solution (pH7.3). The sample in contact with ADL-Alexa488 and the Alexa488 loading control was incubated at room temperature for one hour. The incubated sample was diluted with BSA and filtered through a 0.22 μM filter membrane before HPLC analysis on a column packed with gel media (Phenomenex BioSep SEC-S3000).

HPLC analysis was performed by running the standards, the high, medium, and low controls and then the processed patient sample. Free ADL-Alexa488 (M_{w}˜150 kDa) eluted at a retention time of approximately 10-11.5 minutes, ATA-ADL-Alexa488 complexes eluted at the range from 6-10 minutes and the deactivated Alexa488 loading control eluted between 13.5-14.5 minutes (FIG. 3C,D).

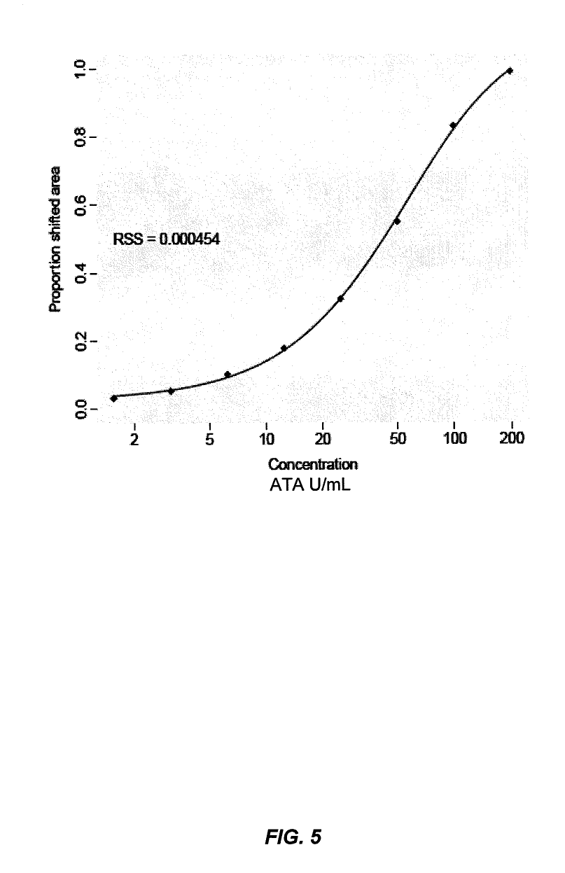

Quantification of ATA and free ADL was performed by tracking the appearance of the high molecular weight peaks (Rₜ=6-10 min) or the disappearance of the free ADL-Alexa488 peak (Rₜ=10-11.5 min). Raw chromatograms were collected in Agilent ChemStation and then exported to the program “R” for automated analysis. Fractions of the shifted area representing ATA-ADL-Alexa488 complexes from different concentrations of added ATA were used to generate an ATA standard curve and fitted with a 5-parameter logistic (5-PL) model to account for asymmetry. Unknowns were determined from the standard curve and given the effective concentration of ATA in 100% serum. FIG. 5 shows an exemplary standard curve for ATA.

### Example 6

### Calculation of Total Amount of Autoantibody to Adalimumab (Total ATA)

When ADL is present in a sample, the total ATA is calculated using the equilibrium equation:

A+B+C=AC+BC, where A=unlabeled adalimumab, B=labeled-adalimumab and C=ATA

In this equation the following values are known for each sample:

A is the concentration from performing the adalimumab mobility shift assay.

B is the known amount of adalimumab-AlexaFluor488 spiked into the sample.

BC is the concentration determined from the ATA mobility shift assay.

Knowing that the sample is acid dissociated and then allowed to reach equilibrium:

$$\frac{BC}{B} = \frac{A C}{A}$$

By solving for AC, the concentration of ATA bound to unlabeled adalimumab is obtained.

Therefore, the total ATA in the sample is then equal to AC+BC.

$$ATI bound to unlabeled IFX = \frac{U \text{/} mL ATA from mobility shift assay \times μg Unlabeled ADL}{μg L abeled ADL}$$

### (a) Calculation of ATI Bound to Unlabeled ADL

Detailed equation for calculation of ATA bound to unlabeled ADL:

$$ATA bound to unlabeled IFX = \frac{\begin{matrix}U \text{/} mL ATA from mobility shift assay \times \\ \begin{matrix}μg \text{/} mL ADL in 100 \% serum \times \\ Vol of 100 \% serum added to ATA assay\end{matrix}\end{matrix}}{\begin{matrix}Volume of labeled ADL added to sample \times \\ Concentration of labeled ADL added to sample\end{matrix}}$$

Exemplary calculation of ATA bound to unlabeled ADL and total ATA for a sample with 100 ug/mL ADL and an ATA mobility shift assay result of 4.2 U/mL ATA.

$$ATA bound to unlabeled ADL = \frac{4.20 U \text{/} mL \times 0.1 mg \text{/} mL \times 0.024 mL}{0.033 mL \times 0.0135 mg \text{/} mL}$$

$$ATA bound to unlabeled ADL = 22.6 U \text{/} mL$$

### (b) Calculation of Total Amount of Autoantibodies (Total ATA)

Calculation of total ATA:

Total ATA=ATA bound to unlabeled ADL+ATA bound to labeled ADL

Exemplary calculation of total ATA:

$$\begin{matrix}Total ATA = 22.6 U \text{/} mL + 4.2 U \text{/} mL \\ = 26.8 U \text{/} mL\end{matrix}$$

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for determining the presence or level of an anti-TNFα drug in a sample, the method comprising:
(a) contacting a labeled TNFα with a sample having an anti-TNFα drug to form a labeled complex with the anti-TNFα drug;
(b) subjecting the labeled complex to size exclusion chromatography to separate the labeled complex from free labeled TNFα and to measure the amount of the labeled complex and the amount of the free labeled TNFα;
(c) calculating a ratio of the amount of the labeled complex to the sum of the labeled complex plus free labeled TNFα; and
(d) comparing the ratio calculated in step (c) to a standard curve of known amounts of the anti-TNFα drug, thereby determining the presence or level of the anti-TNFα drug.

2. The method of claim 1, wherein the standard curve is generated by incubating the labeled TNFα with known amounts of the anti-TNFα drug.

3. The method of claim 1, wherein the standard curve has a y-axis comprising the ratio of labeled complex to the sum of the amount of the labeled complex plus free labeled TNFα and an x-axis comprising known amounts of anti-TNFα drug.

4. The method of claim 1, wherein the sample is serum.

5. The method of claim 1, wherein the anti-TNFα drug is a member selected from the group consisting of REMICADE™ (infliximab), ENBREL™ (etanercept), HUMIRA™ (adalimumab), CIMZIA® (certolizumab pegol), and combinations thereof.

6. The method of claim 1, wherein the size exclusion chromatography is size exclusion-high performance liquid chromatography (SE-HPLC).

7. The method of claim 1, wherein the labeled TNFα is a fluorophore-labeled TNFα.

8. The method of claim 7, wherein the fluorophore is an Alexa Fluor® dye.

9. The method of claim 1, wherein the labeled complex is eluted first, followed by the free labeled TNFα.

10. The method of claim 1, wherein the sample is obtained from a subject receiving therapy with the anti-TNFα drug.

11. A method for determining the presence or level of an autoantibody to an anti-TNFα drug in a sample, the method comprising:
(a) contacting a labeled anti-TNFα drug with the sample to form a labeled complex with the autoantibody;
(b) subjecting the labeled complex to size exclusion chromatography to separate the labeled complex from free labeled anti-TNFα drug and to measure the amount of the labeled complex and the amount of the free labeled anti-TNFα drug;
(c) calculating a ratio of the amount of the labeled complex to the sum of the amount of the labeled complex plus free labeled anti-TNFα drug; and
(d) comparing the ratio calculated in step (c) to a standard curve of known amounts of the autoantibody, to thereby determine the presence or level of the autoantibody.

12. The method of claim 11, wherein the standard curve is generated by incubating the labeled anti-TNFα drug with serum positive for the autoantibody.

13. The method of claim 11, wherein the standard curve has a y-axis comprising the ratio of the amount of labeled complex to the sum of the amount of the labeled complex plus free labeled anti-TNFα drug and an x-axis comprising known amounts of the autoantibody.

14. The method of claim 11, wherein the sample is serum.

15. The method of claim 11, wherein the sample is incubated with acid prior to admixing labeled anti-TNFα drug to dissociate any unlabeled anti-TNFα drug and autoantibody complex.

16. The method of claim 11, wherein the anti-TNFα drug is a member selected from the group consisting of REMICADE™ (infliximab), ENBREL™ (etanercept), HUMIRA™ (adalimumab), CIMZIA® (certolizumab pegol), and combinations thereof.

17. The method of claim 11, wherein the autoantibody is a member selected from the group consisting of a human anti-mouse antibody (HAMA), a human anti-chimeric antibody (HACA), a human anti-humanized antibody (HAHA), and combinations thereof.

18. The method of claim 11, wherein the size exclusion chromatography is size exclusion-high performance liquid chromatography (SE-HPLC).

19. The method of claim 11, wherein the labeled anti-TNFα drug is a fluorophore-labeled anti-TNFα drug.

20. The method of claim 19, wherein the fluorophore is an Alexa Fluor® dye.

21. The method of claim 11, wherein the labeled complex is eluted first, followed by the free labeled anti-TNFα drug.

22. The method of claim 11, wherein the sample is obtained from a subject receiving therapy with the anti-TNFα drug.

23. The method of claim 11, wherein alternatively, a ratio of the free labeled anti-TNFα drug to an internal control is determined and used to extrapolate the level of the autoantibody from the standard curve.

24. A method for determining the total amount of autoantibody in a sample, the method comprising:
(a) determining the level of autoantibody by:
(i) contacting a labeled anti-TNFα drug with the sample to form a labeled complex with the autoantibody;
(ii) subjecting the labeled complex to size exclusion chromatography to separate the labeled complex from free labeled anti-TNFα drug and to measure the amount of the labeled complex and the amount of the free labeled anti-TNFα drug;
(iii) calculating a ratio of the amount of the labeled complex to the sum of the amount of the labeled complex plus free labeled anti-TNFα drug;
(iv) comparing the ratio calculated in step (c) to a standard curve of known amounts of the autoantibody, to thereby determine the presence or level of the autoantibody, bound to a labeled anti-TNFα drug; and
(b) adding the amount of autoantibody bound to unlabeled anti-TNFα drug to the level determined in step (a) to produce the total amount of autoantibody in the sample.

25. The method of claim 24, wherein the amount of autoantibody bound to unlabeled anti-TNFα drug is calculated by multiplying the level of autoantibody bound to labeled anti-TNFα drug of step (a) by the amount of unlabeled anti-TNFα drug divided by the amount of labeled anti-TNFα drug.

26. The method of claim 25, wherein the amount of unlabeled anti-TNFα drug is the weight of anti-TNFα drug determined by multiplying the concentration of anti-TNFα drug by the volume of sample.

27. The method of claim 25, wherein the amount of labeled anti-TNFα drug is the weight of labeled anti-TNFα drug determined by multiplying the volume of labeled anti-TNFα drug by the concentration of labeled anti-TNFα drug added to the sample.
